(19) United States
(12) Patent Application Publication
(10) Pub. No.: US 2021/0378949 A1
(43) Pub. Date: Dec. 9, 2021

(54) BIODEGRADABLE ANTIBACTERIAL PIEZOELECTRIC WOUND DRESSING

(71) Applicant: University of Connecticut, Farmington, CT (US)

(72) Inventors: Thanh Duc Nguyen, South Windsor, CT (US); Eli Curry, Franklin, CT (US); Ritopa Das, West Springfield, MA (US); Yang Liu, Willimantic, CT (US)

(21) Appl. No.: 17/342,470

(22) Filed: Jun. 8, 2021

Related U.S. Application Data
(60) Provisional application No. 63/035,891, filed on Jun. 8, 2020.

(51) Int. Cl. A61K 9/00; H01L 41/45; A61L 15/42; A61K 9/70; A61L 15/64; A61L 15/26; C08L 67/04

(52) U.S. Cl. CPC A61K 9/0009; H01L 41/45; A61L 15/425; B82Y 5/00; A61L 15/64; A61L 15/26; C08L 67/04; A61K 9/70

(57) ABSTRACT

Methods, systems, and apparatus, including combination therapy systems for the treatment of infections. These systems comprising: an ultrasound device capable of producing ultrasonic acoustic pressure; and a biodegradable piezoelectric film comprising polymer-based piezoelectric material, poly(L-lactic acid) (PLLA) nanofiber mesh configured to, when placed in an electrolytically conductive environment comprising water and stimulated with the ultrasonic acoustic pressure, vibrate generating electricity to locally decomposes the water into reactive oxygen species (ROS) to induce a broad-spectrum bactericidal effect.

### CROSS-REFERENCE TO RELATED APPLICATIONS

This application claims the benefit of U.S. Provisional Patent Application No. 63/035,891, filed Jun. 8, 2020, the entire content of which is hereby incorporated by reference.

### FEDERALLY SPONSORED RESEARCH

This invention was made with United States government support under National Institutes of Health grant number AR076646. The United States government has certain rights in the invention.

### BACKGROUND

The development of alternate therapeutic approaches for the treatment of antibiotic-resistant infections is a continuously expanding field of research. While recent progress has been made in electroceutical nanotechnology, and photolysis-based approaches, most of these fields have limited efficacy against antibiotic-resistant infections or are dependent upon technology that currently does not exist in a clinical setting.

### SUMMARY

Antibiotic resistance is rapidly becoming a major threat to the United States. In 2019 the Centers for Disease Control and Prevention (CDC) reported 2.8 million antibiotic-resistant infections occurred in the United States alone, which resulted in more than 35,000 deaths. According to the CDC's report, multidrug-resistant Pseudomonas aeruginosa (P. aeruginosa) and Methicillin-resistant Staphylococcus aureus (MRSA) are considered serious threats. Both bacteria are becoming increasingly prevalent in hospital-acquired infections, and in 2017 it was estimated that 323,700 patients were hospitalized with MRSA and another 32,600 patients were hospitalized due to P. aeruginosa infections. Due to this ongoing threat, researchers have proposed many approaches for the treatment of P. aeruginosa and MRSA.

The use of antibiotics remains the traditional approach by doctors for the prevention or treatment of bacterial infections. Antibiotics tend to fall under two categories: bacteriostatic (preventing bacterial growth) or bactericidal (killing bacteria). Researchers originally believed that bactericidal antibiotics-initiated cell death via three main macromolecular targets: cell wall biosynthesis inhibition, protein synthesis inhibition, or DNA gyrase inhibition. However, recent research indicates that drugs, regardless of macromolecular targets, initiate cell death by inducing intracellular production of hydroxyl radicals. While there are still several drugs that are available to treat P. aeruginosa and MRSA infections, the number of available drug options are reducing due to multidrug-resistance and reduced inventions by the pharmaceutical industry. Additionally, the use of antibiotics to treat P. aeruginosa and MRSA infections is not without its pitfalls. Antibiotics are unable to effectively lyse bacteria encased in biofilms, which immediately reduce their efficacy against P. aeruginosa and MRSA, both of which are known to form biofilms. Many bacteria have also developed the ability to pump antibiotics out of their intracellular space or enzymatically break them down prior to their generation of hydroxyl radicals. Additionally, the use of strong antibiotics like clindamycin can have adverse side effects such as Clostridium difficile-associated diarrhea due to non-targeted delivery and broad-spectrum effects on gut bacteria. Therefore, there exists a need to develop an approach that can break apart biofilms while locally delivering hydroxyl radicals to induce bacterial cell death.

Due to issues with antibiotics, researchers have been investigating alternative approaches to treating bacterial infections using nanotechnology, electroceuticals and photolysis. Copper, silver and iron oxide nanoparticles are becoming widely used in research and commercial grade wound dressings due to their antimicrobial properties. While these approaches show promise for their antimicrobial properties, metal nanoparticles can only be used in minute quantities, due to their cytotoxic effects on mammalian cells. Electroceuticals use electricity to stimulate the regeneration of tissue as well as inhibit bacterial growth in wounds via reactive oxygen species (ROS) or heat production. While electroceuticals are slowly becoming commercially available, they have similar issues to nanoparticle-based approaches. Specifically, electroceuticals employ the use of potentially cytotoxic metals in non-degradable or partially degradable devices. Additionally, electroceuticals are traditionally designed for open surface wounds, rendering them useless for deep surgical wound treatment. Photolysis based technology employs a photosensitive dye (either injected or naturally present in the bacteria) and light to disrupt the cell membrane, which makes the bacteria susceptible to ROS-mediated death. Depending on the wavelength of light used in the procedure, treatment of infections can happen at depths up to 1 cm. However, this renders the technology limited in its treatment of deep surgical site infections. Therefore, a technology platform that can safely treat both topical and deep infections all while utilizing safe materials is clinically needed.

Accordingly, implementations of the present disclosure are generally directed to biodegradable piezoelectric polymer-based piezoelectric material, poly(L-lactic acid) (PLLA) nanofiber mesh that can, when stimulated with ultrasound (US), generate ROS to treat antibiotic-resistant infections. Additionally, the use of US can break apart bacterial biofilms and increase cell membrane permeability for, for example, P. aeruginosa and MRSA while stimulating the PLLA nanofibers to generate ROS and induce a broad-spectrum bactericidal effect. The charge generated via the use of US can also recruit cells and facilitate tissue skin healing, thus offering an exemplary material for wound healing and tissue skin regeneration. In some embodiments, the piezoelectric PLLA nanofiber mesh comprises biodegradable piezoelectric materials that includes PLLA, silk, glycine composites, and so forth. The described combination therapy system employing US to stimulate biodegradable piezoelectric PLLA nanofiber mesh (PLLA+US) provides a drug-free approach and a safe platform to effectively treat antibiotic-resistant infections in any location of the body.

Particular implementations of the subject matter described in this disclosure can be implemented so as to realize one or more of the following advantages. The described combination therapy system is highly translational for clinical use and can be seamlessly integrated into preexisting medical procedures for the treatment of antibiotic-resistant infections. For example, piezoelectric PLLA nanofiber mesh can be implanted inside the body prior to closure of a surgical site defect or used to dress open wounds. In some embodiments, once the device is stimulated with non-invasive US, is vibrates generating small amounts of electricity (e.g., between about 50 and 75 megavolts (mV) in response to a 40 Kilohertz (kHz) US treatment). The electricity locally decomposes water into reactive oxygen species, which lyse bacteria and sterilize the area around the mesh (e.g., within several centimeters up to around a meter from the mesh). Moreover, the piezoelectric PLLA nanofiber mesh self-degrades after a pre-defined lifetime without the need for an invasive removal surgery, which facilitates cell ingrowth and tissue skin regeneration. Additionally, when compared to preexisting treatment options, the described combination therapy system offers a safer drug and metal free approach for the treatment of antibacterial infections. The described combination therapy system can reduce the potential for side effects when compared to available antibiotics due to the localized delivery of reactive oxygen species. The described combination therapy system can also remove the potential for cytotoxic effects caused by metals used in electroceutical and nanoparticle-based designs. The biodegradable nature of the piezoelectric PLLA nanofiber mesh allows it to be used in both superficial and deep wounds, something current electroceutical designs cannot do. Additionally, piezoelectric PLLA nanofiber mesh net cost is cheaper than electroceuticals because no metals are involved in the design of this product. Thus, in conjunction with the antibacterial properties, the described combination therapy system employing PLLA+US accelerates wound healing while resulting in no harm to the body.

The described combination therapy system may be employed by, for example, organizations developing wound dressings for cuts or burns, organizations developing metal implants (e.g., incorporated as a surface coating to sterilize the device post-implantation), organizations developing electroceuticals (e.g., used as a battery-free approach to electrically-stimulated wound repair), patients with superficial burns and cuts, patients with deep wounds (e.g., infected surgical site defects), medical professional treating patients with deep or superficial wounds, and so forth.

It is appreciated that methods in accordance with the present disclosure can include any combination of the aspects and features described herein. That is, methods in accordance with the present disclosure are not limited to the combinations of aspects and features specifically described herein, but also may include any combination of the aspects and features provided.

The details of one or more implementations of the present disclosure are set forth in the accompanying drawings and the description below. Other features and advantages of the present disclosure will be apparent from the description and drawings, and from the claims.

### BRIEF DESCRIPTION OF DRAWINGS

FIG. 1 depicts an example system 100 employing the described PLLA nanofiber mesh to lyse bacteria.

FIGS. 2A-2C depicts information regarding the physical characterization of the piezoelectric PLLA nanofiber mesh.

FIGS. 3A-3D depict the effect of using US to stimulate biodegradable piezoelectric PLLA nanofiber mesh.

FIG. 4 depicts a generalized process to fabricate a piezoelectric PLLA mesh such as can be employed in the described combination therapy system.

FIG. 5 depicts a simplified schematic for mice undergoing US therapy.

FIGS. 6A-6C depict fibroblast proliferation and migration on the piezoelectric PLLA nanofiber mesh.

FIG. 7 depicts a flow diagram an example process for lysing bacteria at a wound site.

### DETAILED DESCRIPTION

Embodiments of the present disclosure are generally directed a combination therapy system for treating various infections in wounds using a film comprised of a biodegradable piezoelectric PLLA nanofiber mesh. More particularly, embodiments of the present disclosure are directed to generating ROS by stimulating a biodegradable piezoelectric PLLA nanofiber mesh with US to treat various infections (e.g., antibiotic-resistant infections).

Piezoelectricity is a phenomenon that refers to a material's ability to generate electricity when mechanically deformed and vice versa. Piezoelectric materials are currently used in a variety of different applications, including sensing, energy harvesting, and US. However, a recently emerging field of research called piezocatalysis relies on mechanically induced charges from piezoelectric materials to catalyze a chemical reaction. Piezocatalysis typically uses a piezoelectric material to drive the redox reaction of water in an electrolytically conductive environment. The redox reaction of water produces ROS, such as .OH or .O₂₋, which are then used to accelerate the decomposition of toxic fluorescent dyes (e.g., Rhodamine B or Acid orange 7 (AO7)) in wastewater. While this research shows promise for environmental applications, it can also be applied to the treatment of antibiotic-resistant infections.

Within the last decade, researchers have been heavily investigating the effects of ROS on bacteria. Bactericidal antibiotics appear to generate ROS and the presence of ROS induces cell death. Even though the therapeutic quantities of ROS necessary to induce cell death and the mechanisms by which ROS induces cell death are still being debated, researchers appear to agree that ROS will cause the death of bacteria. Therefore, using piezocatalysis to lyse bacteria via ROS is a possible approach to treating bacterial infections. However, previously reported materials used for piezocatalysis (e.g., Zinc Oxide (ZnO) or Barium Titanate (BaTiO3)) are either toxic and/or non-degradable, thereby limiting their applications.

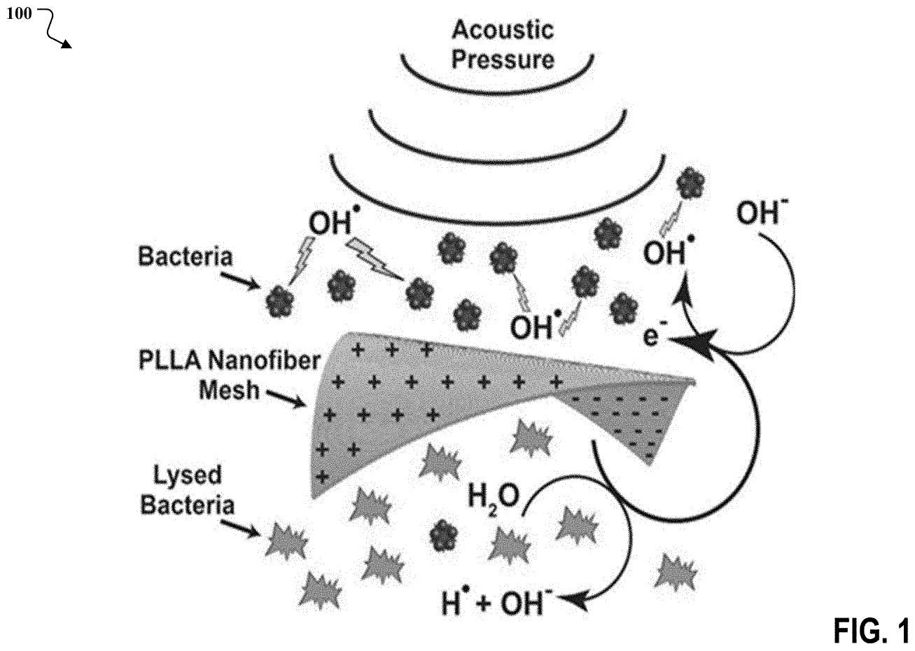

Accordingly, the described combination therapy system's use of US to stimulate biodegradable piezoelectric PLLA nanofiber mesh generating ROS to lyse bacteria (see FIG. 1) offers doctors a drug-free alternative to dress and sterilize both open wounds and burns as well as surgical site defects that can degrade away after a controlled period. While electroceuticals have been used to treat bacterial infections, their applications in a clinical setting are limited due to their reliance on potentially cytotoxic metals and non-degradable batteries. However, the piezoelectric PLLA nanofiber mesh in conjunction with non-invasive US offers a wireless electroceutical approach that avoids the use of metals and external power sources. Additionally, using the PLLA to drive the redox reaction of water under applied US allows the material to generate ROS species, like .OH, .O₂₋, and H₂O₂, which have the same therapeutic efficacy as commercially available antibiotics.

FIG. 1 depicts an example system 100 employing the described PLLA nanofiber mesh to lyse bacteria. As depicted, ultrasonic acoustic pressure (e.g., US) is applied to the PLLA nanofiber mesh, which causes a deformation. This deformation generates a charge that drives the redox reaction of water. The resulting hydroxyl radicals then attack the bacteria, induce bacterial cell death. Moreover, the PLLA nanofiber mesh may be employed as a piezoelectric-based implant in a patient that degrades away after the successful treatment of bacterial infections. Thus, avoiding the need for future corrective surgeries.

As can be seen in FIG. 1, as the 14 mm×14 mm×20 μm piezoelectric PLLA nanofiber mesh is subjected to between (40±4 Kilohertz (kHz)) US, the applied acoustic pressure causes the film to bend, which generates electricity when the mesh is placed in an electrolytically conductive environment, such as salt water or bodily fluids. The generated electricity drives a local redox reaction for water, causing it to decompose and form ROS. Moreover, the US can break apart bacterial biofilms and increase cell membrane permeability in the bacteria, all while stimulating the PLLA nanofibers to locally generate ROS to induce a broad-spectrum bactericidal effect. Generally, any ultrasound capable device will generate electric charges; however, the amount of charge will vary depending on ultrasound frequency and intensity.

### Physical Characterizations of the PLLA Nanofiber Mat to Serve as a Good Wound Dressing Material

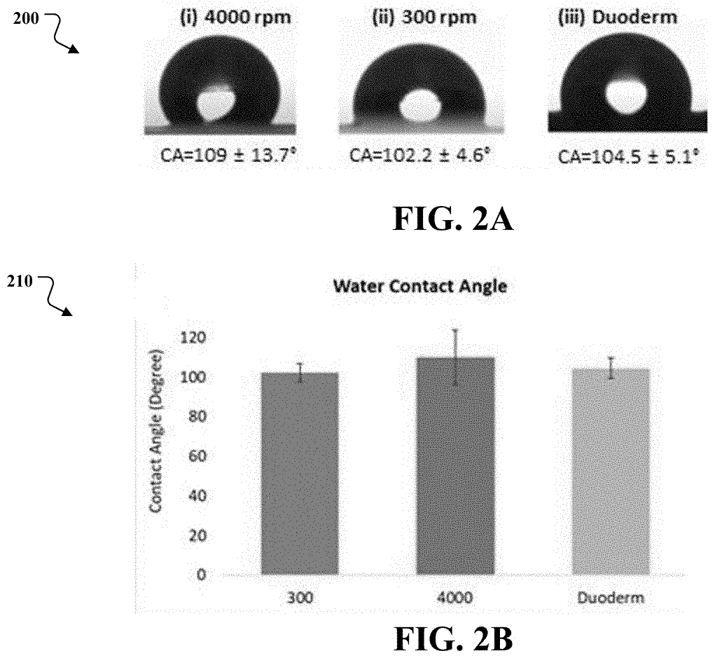

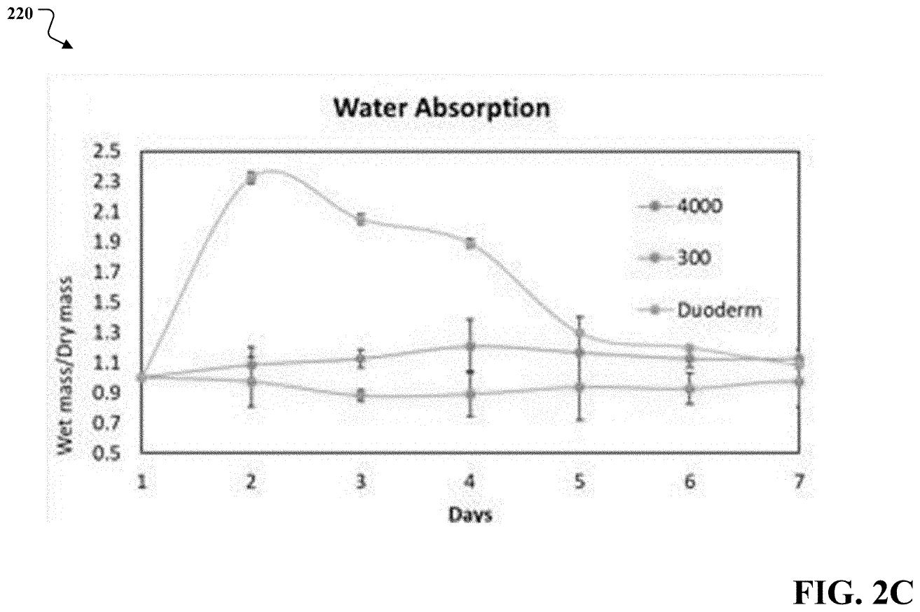

FIGS. 2A-2C depicts information regarding the physical characterization of the piezoelectric PLLA nanofiber mesh showing that the material can be employed as a dressing for wound care. FIG. 2A depicts optical images 200 of a drop of water on the piezoelectric PLLA nanofiber mesh as collected from the goniometer. FIG. 2B depicts a graph 210 that includes a quantitative representation of the contact angles on a 4000-rpm film, 300-rpm film and duoderm, indicating no significant difference between them. FIG. 2C depicts a graph 220 that includes the water absorption in on a 4000-rpm film, 300-rpm film and duoderm indicating that both electrospun films absorb very little water whereas duoderm absorbs a much higher amount.

In one experiment, to study the physical characteristics of the piezoelectric PLLA nanofiber mesh, contact angle measurements and water absorption measurements were performed on the material. In both cases the results for the piezoelectric film (4000 rpm) were compared with those of the non-piezoelectric film (300 rpm) and duoderm, a commercial grade wound dressing. The contact angle measurements were performed using a Rame Hart Model 100 Goniometer. As seen in FIGS. 2A and 2B, the contact angles of all three materials were in the range of 100-110° indicating that each material is slightly hydrophobic. Additionally, there was no statistically significant difference in the contact angles of any of the materials as was determined by using a student t-test. For measuring the water absorption, each material was cut into 0.5″×0.5″ sized pieces and weighed. The pieces were immersed in deionized water and maintained at 37° C. in an incubator. The weights of the materials were recorded every day. As seen in FIG. 2C, both the 4000 rpm (i.e. piezoelectric) and 300 rpm (i.e. non-piezoelectric) films had almost no swelling with a wet mass/dry mass ratio close to 1.

However, the duoderm demonstrated a much higher swelling ratio, possibly due to the presence of a highly porous sponge-backing layer. As such the slight hydrophobicity, low water absorption and retention by the piezoelectric PLLA nanofiber mesh will further prevent bacterial infection by keeping the wound site dry.

### Effect of Using US to Stimulate Biodegradable Piezoelectric PLLA Nanofiber Mesh

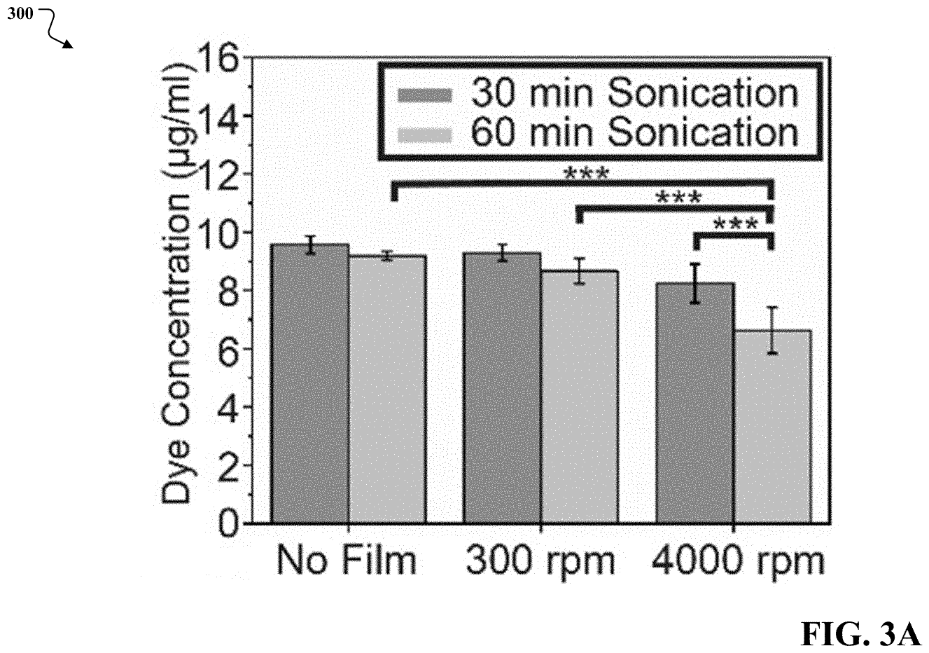

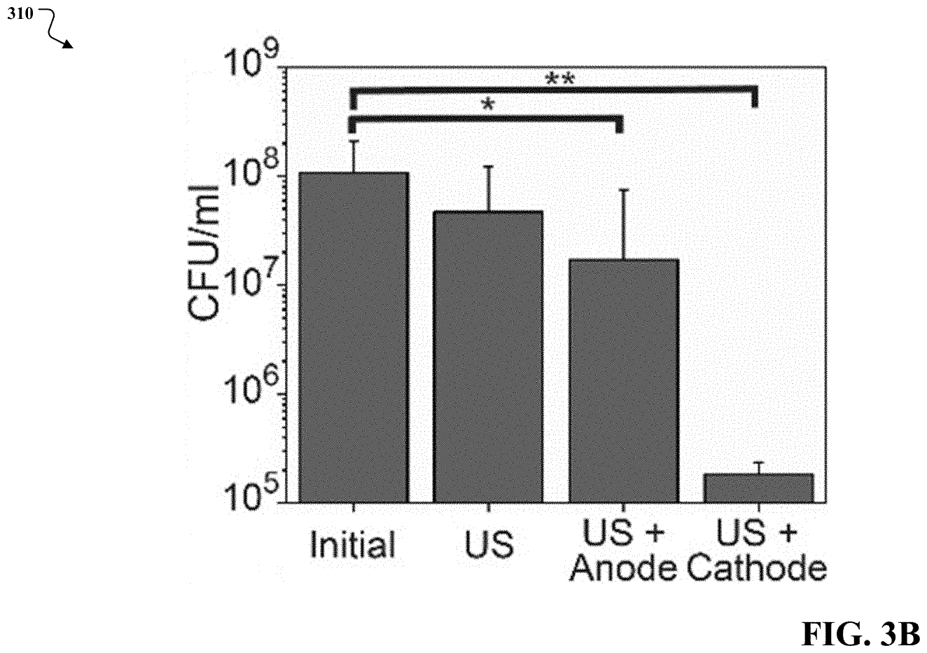

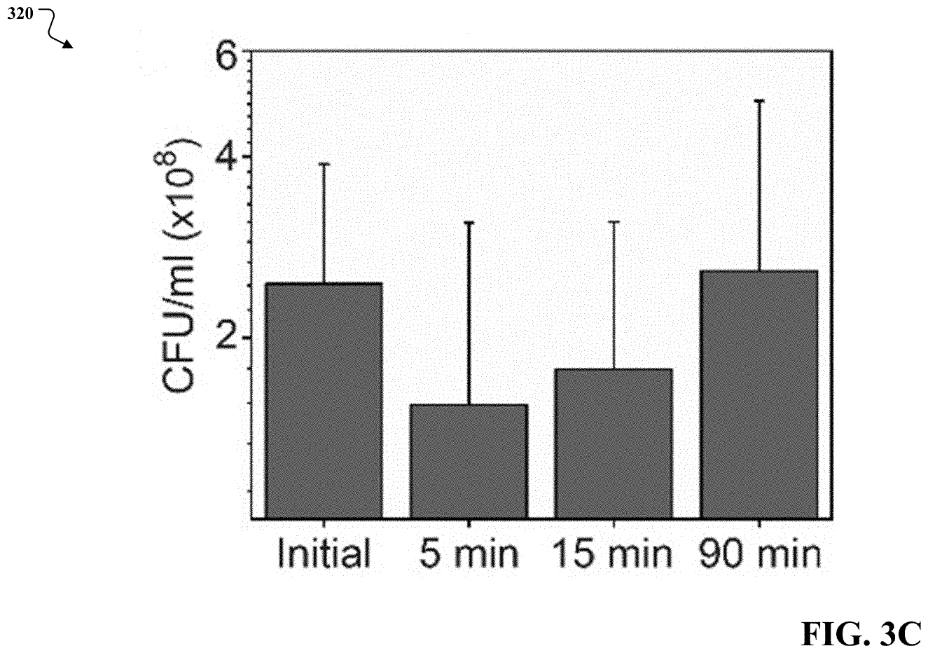

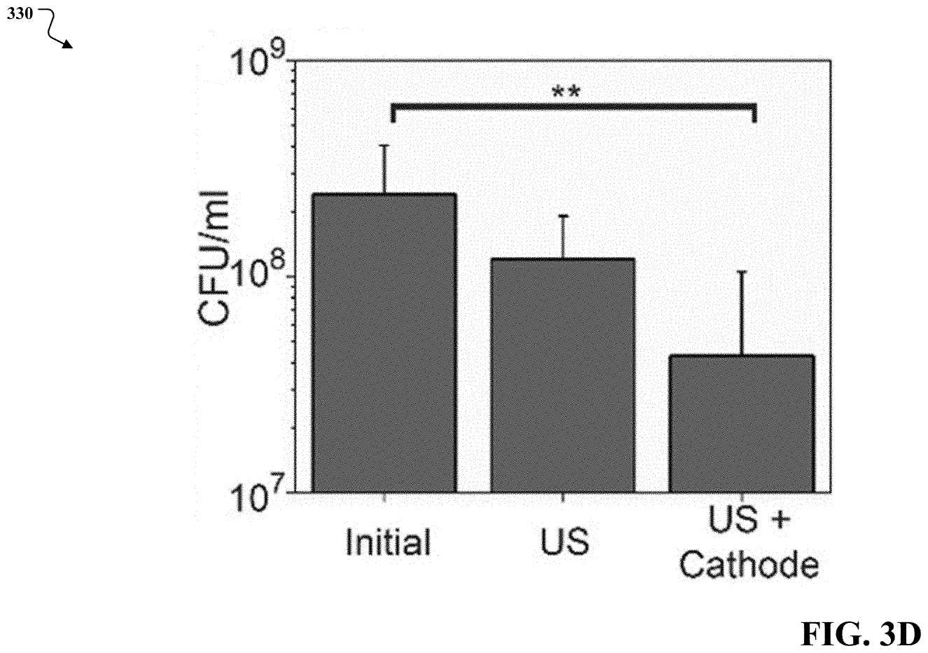

FIGS. 3A-3D depict the effect of using US to stimulate biodegradable piezoelectric PLLA nanofiber mesh (piezoelectric PLLA+US) on Sulforhodamine B dye as well as P. aeruginosa and S. aureus. FIG. 3A depicts a graph 300 showing that the piezoelectric PLLA (4000 rpm)+US can significantly decompose Sulforhodamine B dye, which is indicative of ROS being generated by the PLLA film (***, p<0.001). FIG. 3B depicts a graph 310 showing that exposing P. aeruginosa (*, p<0.05) and (**, p<0.01) to piezoelectric PLLA+US for 90 minutes has a significant effect.

FIG. 3C depicts a graph 320 showing that the use of US at 40 kHz breaks apart S. aureus biofilms, resulting in an increase in cell concentration over time. FIG. 3D depicts a graph 330 exposing S. aureus (**, p<0.01) to piezoelectric PLLA+US for 90 minutes has a statistically significant effect.

Previous research in the field of piezocatalysis has shown that fluorescent dyes decompose in the presence of ROS. In one example, the PLLA nanofiber mesh was submerged in an electrolytically conductive solution of saltwater and sulforhodamine B dye to determine if the piezoelectric PLLA nanofiber mesh generates ROS when subjected to 40 kHz US. A test tube containing the dye solution and film was lowered into an ultrasonic water bath. As show in FIG. 3A, the piezoelectric PLLA nanofiber mesh (4000 rpm) has a statistically significant effect (p<0.001) on the decomposition of the fluorescent dye. Crystallinity and orientation of the polymer chains are two material properties that provide for piezoelectricity in PLLA. The highly aligned and crystalline 4000 rpm sample has significantly stronger piezoelectric properties when compared to the unoriented and less crystalline 300 rpm sample. Therefore, a weaker piezoelectric PLLA film (300 rpm) and just the dye in the presence of US were used as controls to illustrate that the 4000-rpm piezoelectric PLLA clearly generates more ROS. Additionally, the quantities of ROS can be tuned based on the duration of the applied US. This is shown in FIG. 3A by the statistically significant difference between the dye concentrations at 30 minutes and 60 minutes of ultrasonic treatment.

In another example, the piezoelectric PLLA was submerged in an electrolytically conductive solution (phosphate buffered saline (PBS)) with roughly 1×10⁸ colony-forming unit (CFU)/milliliter (ml) of P. aeruginosa (strain: Boston 41501) and Staphylococcus aureus (S. aureus) (strain: Wichita) to illustrate the antibacterial effects. As shown in FIG. 3B, the piezoelectric PLLA has a statistically significant effect on P. aeruginosa (p<0.01). Additionally, in some examples, the cathode side of the film lyses the bacteria more effectively than the anode side, which is in strong agreement with the proposed operating mechanism illustrated in FIG. 1 showing that the ROS is produced on the cathode side of the PLLA film. However, both cell lines responded differently to the US treatment. For example, an increase in cell concentration is shown in (FIG. 3C) as US is applied to the S. aureus solution for longer periods of time because the gram-positive S. aureus possess a more crosslinked biofilm when compared to the P. aeruginosa.

In another example, as shown in FIG. 3D the initial S. aureus suspension was pre-treating with US for 90 minutes to get it to a planktonic state so that the bacteria were lysed using the combined therapy of piezoelectric PLLA+US after an additional 90 minutes of US treatment. This example illustrates the piezoelectric PLLA material's ability to lyse both gram-negative and gram-positive bacteria and reinforces the employment of the described combination therapy system as an alternative to broad-spectrum antibiotics.

### ROS Species Generated by Piezoelectric PLLA Nanofibers

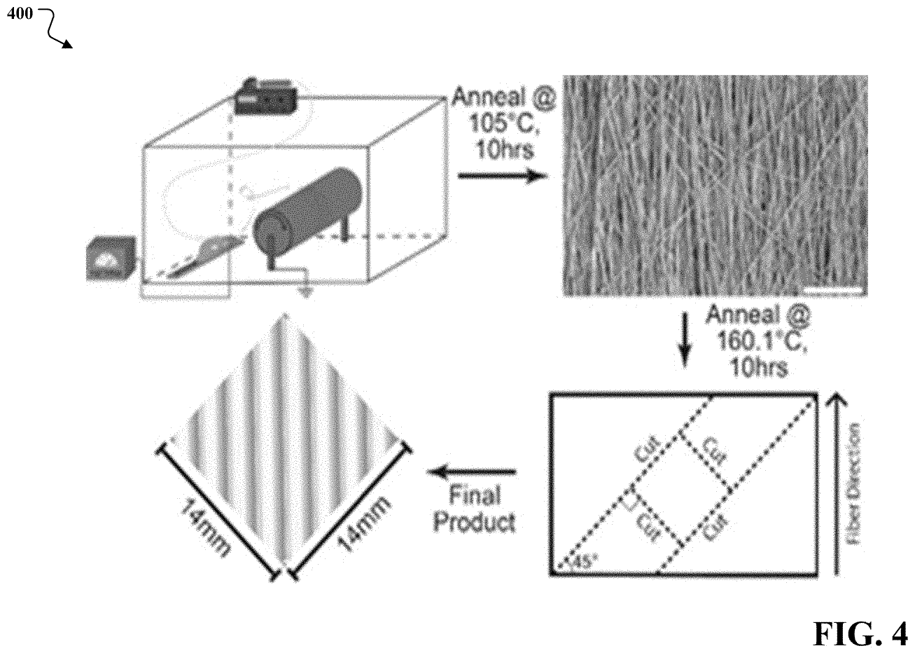

FIG. 4 depicts a generalized process 400 to fabricate a piezoelectric PLLA mesh such as can be employed in the described combination therapy system. Process 300 depicts an aligned, nanofibrous PLLA mesh that initially made using an electrospinning box. After the fibers are annealed, they are cut at an angle (e.g., 45°), resulting in the piezoelectric PLLA mesh. (SEM image scale bar=50 μm). In some examples, the PLLA nanofibers are made by dissolving 0.8 g of poly(L-Lactic acid) in a 1:4 v/v mixture of N, N-Dimethylformamide (DMF, anhydrous, >99.9%) and dichloromethane (DCM) respectively. A solution flow rate is set to 2 ml/hour (hr) through a flat-tipped 22-gauge needle with 14 kV (kilovolts) applied to it. The polarized solution is sprayed at a grounded aluminum drum, wrapped in aluminum foil, rotating at 4,000 rpm (rotations per minute). The fibers are produced in a 40±15% relative humidity atmosphere at ambient temperature resulting in a piezoelectric PLLA nanofiber mesh comprised of highly aligned nanofibers. These fibrous meshes are annealed at 105° C. for 10 hr after which the oven is shut off and allowed to cool to room temperature. The fibrous meshes are peeled off the aluminum foil and transferred to a Teflon FEP sheet. Another FEP sheet is placed on top of the film and the sandwiched film is placed on top of a glass slide. The sandwiched film is be placed inside an oven at 160.1° C. for 10 hr after which the oven is shut off and allowed to cool to room temperature. The electro spun films is cut at an angle (e.g., 45°) relative to the oriented direction to harvest the shear piezoelectric signal of the film. In such example, the final device has relative dimensions of about (14 millimeters (mm)×14 mm×20 micrometers (μm)).

The influence of zeta potential on the antimicrobial properties of materials has shown that a positive surface charge imparts inherent antimicrobial properties for metal nanoparticles. Accordingly, in one example, a Zetasizer Nano, with a surface zeta potential cell, was used to measure the surface charge for the piezoelectric PLLA nanofiber mesh. After preparing a solution with a known concentration of electrospun PLLA dissolved in deuterated chloroform, a Bruker DMX 500-megahertz (MHz) NMR spectrometer was used to measure any residual quantities of DCM or DMF. To determine the hydrophobicity or hydrophilicity, a factor that has been shown to influence bacterial adhesion, the contact angle for the piezoelectric PLLA nanofiber mesh was measured using a goniometer. The porosity of the piezoelectric PLLA nanofiber mesh was also measured using a mercury intrusion porosimeter. This measurement shows how fluids move through the PLLA membrane, which drives the redox reaction of water.

To ensure that the proposed piezoelectric PLLA nanofiber mesh can generate charge under the applied 40 kHz US stimulus, the resonance frequency and operational bandwidth for the piezoelectric device need to be determined. Accordingly, the resonance frequency and operational bandwidth for the piezoelectric device for the piezoelectric PLLA nanofiber mesh was measured using a Bode 100 vector network analyzer and a Polytec PSV-500 scanning vibrometer. For both measurement setups, the device dimensions were controlled with 14 mm×14 mm×20 μm for the PLLA film dimensions and electrode dimensions of 12 mm×12 mm×8 μm (deposited by screen printing silver paste). The network analyzer was employed to determine the electro-mechanical resonance frequency by measuring the impedance. As the frequency of the input signal changed, the vector network analyzer measured the impedance of the device and the phase angle of the transmitted signal. By plotting out the impedance and phase angle values over a wide frequency range (1 Hz-500 kHz) the frequency ranges for the planar and thickness resonance modes of the material were determined. The thickness of the PLLA film was changed in 5 μm increments to tune the resonance frequency (defined as the first frequency in the impedance spectrum with a local minima for an impedance value) to be within 4 kHz of the 40 kHz ultrasonic input. This allowed for maximum charge output while maintaining the mechanical integrity of the device under the applied 40 kHz ultrasonic stimulus. After tuning the thickness of the device to achieve the desired resonance frequency (40±4 kHz), the scanning vibrometer was employed to confirm the accuracy of the initial measurement. A voltage waveform with a controlled amplitude (500 Vpp) and varying the frequency (25-50 kHz) was applied while simultaneously measuring the displacement using the vibrometer to detect the resonance frequency for the film (denoted by the frequency at which maximum displacement occurs). Together both experiments will be used to ensure the device optimally performs as an antibacterial wound dressing.

In some embodiments, the desired resonance frequency of the ultrasonic stimulus comprises between about 10 kHz and about 70 kHz. In some embodiments, the desired resonance frequency of the ultrasonic stimulus comprises between about 20 kHz and about 60 kHz. In some embodiments, the desired resonance frequency of the ultrasonic stimulus comprises between about 30 kHz and about 50 kHz. In some embodiments, the desired resonance frequency of the ultrasonic stimulus comprises between about 5 kHz and about 40 kHz. In some embodiments, the desired resonance frequency of the ultrasonic stimulus comprises between about 40 kHz and about 80 kHz. In some embodiments, the desired resonance frequency of the ultrasonic stimulus comprises between about 5 kHz, about 10 kHz, about 15 kHz, about 20 kHz, about 21 kHz, about 22 kHz, about 23 kHz, about 24 kHz, about 25 kHz, about 26 kHz, about 27 kHz, about 28 kHz, about 29 kHz, about 30 kHz, about 31 kHz, about 32 kHz, about 33 kHz, about 34 kHz, about 35 kHz, about 36 kH, about 37 kHz, about 38 kHz, about 39 kHz, about 40 kHz, about 41 kHz, about 42 kHz, about 42 kHz, about 44 kHz, about 45 kHz, about 46 kHz, about 47 kHz, about 48 kHz, about 49 kHz, about 50 kHz, about 51 kHz, about 52 kHz, about 53 kHz, about 54 kHz, about 55 kHz, about 56 kHz, about 57 kHz, about 58 kHz, about 59 kHz, about 60 kHz, about 60 kHz, about 65 kHz, about 70 kHz, about 75 kHz, or about 80 kHz.

The type and quantity of ROS species being generated by the piezoelectric PLLA nanofiber mesh was investigated to understand the antibacterial mechanism. Specifically, different ROS species generated by the piezoelectric PLLA nanofiber mesh (e.g., the Amplex Red hydrogen peroxide assay, hydroxyphenyl fluorescein (HPF), and dihydroethdium) were used to detect the presence of selective ROS indicators (e.g., hydrogen peroxide (H₂O₂), the hydroxyl radial (.OH) and the superoxide anion (.O₂₋) respectively). The piezoelectric PLLA nanofiber mesh was placed in a 1.5 ml centrifuge tube containing 1 ml of 7.4 pH PBS (1×) supplemented with 10 μM HPF or dihydroethidium (due to the instability of the hydroxyl radical and superoxide anion) for the amplex red assay. The centrifuge tubes were capped and sonicated for periods of 0, 15, 30, 60, 90, 120 minutes in an ultrasonic water bath placed in a dark room. Change in fluorescence for each of the selective dyes was detected using a microplate reader to illustrate the increase in each of the ROS species previously mentioned. A Bruker EMX nano for electron paramagnetic resonance (EPR) spectroscopy was also used to acquire a concentration for each of the ROS species overtime. The test was repeated in a similar manner to the dye tests mentioned above; however, the PLLA film was immersed in 980 μL of PBS (1×) and 20 μL of 5,5-dimethyl-1-pyrroline-N-oxide (DMPO) and then sonicated for 0, 15, 30, 60, 90, 120 minutes.

### Antibacterial Properties of the Piezoelectric PLLA Nanofibers In Vitro

In some embodiments, the treatment with the combination therapy system employing PLLA+US is conducted for between 30-120 minutes in order to lyse planktonic suspensions of P. aeruginosa and S. aureus. While the proposed combination therapy system includes the use of low frequency US and piezoelectric PLLA, heat dissipation to the local environment due to US can influence bacterial survivability. Therefore, it is critical to understand the effect that heat has on the viability of the control strains P. aeruginosa (strain: Boston 41501) and S. aureus (strain: Wichita) to determine the degree to which heat should be included as part of the combination therapy system.

Accordingly, three experiments were performed: a study where the temperature was maintained at 20° C. through the addition of ice to the water bath, another study where the temperature was heated and maintained at 37° C., and finally, a study where the water bath was initially heated to 37° C. and allowed to warm naturally throughout the US treatment. For all three studies, a solution containing 1×10⁸ CFU/ml of bacteria suspended in 1 ml of 7.4 pH PBS (1×) was prepared in a 1.5 ml centrifuge tube. The cells were sonicated in a Branson CPXH ultrasonic water bath, under the previously described temperature conditions, for a period of 0, 15, 30, 60, 90, 120 minutes. For each time point a sample size of (n=9) was used, and all three studies were performed using both control strains. After the bacterial suspensions are treated, 200 μL of each solution was removed and used in a dilution series, where the stock solution was diluted an order of magnitude up to seven times. 5 μL of each dilution was plated in triplicate on a gridded lysogeny broth and agar plate. The plates were inverted and placed in an incubator at 37° C. for 12 hours. The colonies at the highest dilution for each group were visually counted, and the remaining CFU/ml for each group were then calculated.

Experiments were performed to optimize the antibacterial effects of the combination therapy system on the control strains P. aeruginosa (strain: Boston 41501) and S. aureus (strain: Wichita). The power density and the waveform generated by the Branson CPXH ultrasonic water bath was measured using a Bruel and Kjaer 8103 hydrophone and 2692 charge amplifier to provide baseline information for the next set of experiments. These experiments were performed using a Branson SFX150 sonifier because it affords the user control over the 40 kHz US waveform, power, and temperature. In one experiment, the effects of the US waveform (e.g., continuous vs. pulsed) with a power of 100 W and temperature at 37° C. that was kept constant was investigated for both control strains of bacteria. Another experiment tested the effects of the US power with the pulsed waveform and temperature at 37° C. that was kept constant on both control strains of bacteria. For these experiments, a solution containing 1×10⁸ CFU/ml of bacteria suspended in 1 ml of 7.4 pH PBS (1×) was prepared in a 1.5 ml centrifuge tube. The experiments contained three groups: bacteria only with no US, bacteria only with US, and the bacteria+the piezoelectric PLLA mesh. For the groups receiving US, the sonifier tip was lowered to a controlled height in the centrifuge tube and each tube received 60 minutes of US treatment. Each group used a sample size of (n=9). After the bacterial suspensions were treated, 200 μL of each solution was removed and used in a dilution series, where the stock solution was diluted an order of magnitude up to seven times. 5 μL of each dilution was plated in triplicate on a gridded lysogeny broth and agar plate. The plates were inverted and placed in an incubator at 37° C. for 12 hours. The colonies at the highest dilution for each group were visually counted and the remaining CFU/ml for each group is then calculated.

TABLE 1
Shows the experimental groups for in vitro and in vivo antibacterial
studies. For Group 5, the antibiotics used to treat P. aeruginosa
and S. aureus was gentamicin and clindamycin, respectively. Antibiotics
were used at four times the minimum inhibitory concentration.

| Group | Description | Purpose |
|---|---|---|
| 1 | Bacteria Only | Initial Cell Count |
| 2 | Bacteria + US | Control |
| 3 | Bacteria + PLLA Mesh | Control |
| 4 | Bacteria + Antibiotic | Control |
| 5 | Bacteria + PLLA Mesh + US | Experimental Group |

In another experiment to determine the duration of treatment for the proposed combination therapy system to eliminate planktonic suspensions of control strain bacteria, the Branson SFX150 sonifier was set to 37° C. The experiment included the five groups listed in Table 1 above and were repeated for P. aeruginosa (strain: Boston 41501) and S. aureus (strain: Wichita). For all groups, a solution containing 1×10⁸ CFU/ml of bacteria suspended in 1 ml of 7.4 pH PBS (1×) was prepared in a 1.5 ml centrifuge tube. Groups 2 and 5 both receive US and the sonifier tip was lowered to a controlled height in the centrifuge tube. The cells were treated for a period of 0, 15, 30, 60, 90, 120 minutes in accordance with their experimental group. For each time point, a sample size of (n=9) was used. After the bacterial suspensions were treated, 200 μL of each solution was removed and used in a dilution series where the stock solution was diluted an order of magnitude up to seven times. 5 μL of each dilution was plated in triplicate on a gridded lysogeny broth and agar plate. The plates were inverted and placed in an incubator at 37° C. for 12 hours. The colonies at the highest dilution for each group were visually counted, and the remaining CFU/ml for each group was calculated.

In another experiment, to illustrate whether ROS is responsible for the antibacterial properties of the combination therapy system, a known electron scavenger (thiourea) was introduced into the PBS to prevent the ROS generated by the PLLA from lysing the bacteria. For this experiment, the Branson SFX150 sonifier was set to 37° C. The experiment also included the five groups listed in Table 1 above and were repeated for P. aeruginosa (strain: Boston 41501) and S. aureus (strain: Wichita). For the groups, a solution containing 1×10⁸ CFU/ml of bacteria suspended in 1 ml of 7.4 pH PBS (1×) supplemented with 100 mM thiourea was prepared in a 1.5 ml centrifuge tube. Groups 2 and 5 both received US and the sonifier tip was lowered to a controlled height in the centrifuge tube. The cells were treated for a period of 0, 15, 30, 60, 90, 120 minutes in accordance with their experimental group. For each time point, a sample size of (n=9) was used. After the bacterial suspensions were treated, 200 μL of each solution was removed and used in a dilution series where the stock solution was diluted an order of magnitude up to seven times. 5 μL of each dilution were plated in triplicate on a gridded lysogeny broth and agar plate. The plates were inverted and placed in an incubator at 37° C. for 12 hours. The colonies at the highest dilution for each group were visually counted and the remaining CFU/ml for each group were calculated.

In another experiment, to determine the duration of treatment for the proposed combination therapy system to eliminate planktonic suspensions of antibiotic-resistant bacteria, the Branson SFX150 sonifier was set to 37° C. The experiment included the five groups listed in Table 1 above and were repeated for (P. aeruginosa (strain: 1077994) and S. aureus (strain: USA300-HOU-MR). For the groups, a solution containing 1×10⁸ CFU/ml of bacteria suspended in 1 ml of 7.4 pH PBS (1×) supplemented with 100 mM thiourea was prepared in a 1.5 ml centrifuge tube. Groups 2 and 5 both received US and the sonifier tip was lowered to a controlled height in the centrifuge tube. The cells were treated for a period of 0, 15, 30, 60, 90, 120 minutes in accordance with their experimental group. For each time point, a sample size of (n=9) was used. After the bacterial suspensions were treated, 200 μL of each solution was removed and used in a dilution series where the stock solution was diluted an order of magnitude up to seven times. 5 μL of each dilution was plated in triplicate on a gridded lysogeny broth and agar plate. The plates were inverted and placed in an incubator at 37° C. for 12 hours. The colonies at the highest dilution for each group were visually counted and the remaining CFU/ml for each group was calculated.

### Significance of Piezoelectric PLLA Nanofibers Through the Treatment of Antibiotic-Resistant Infections In Vivo

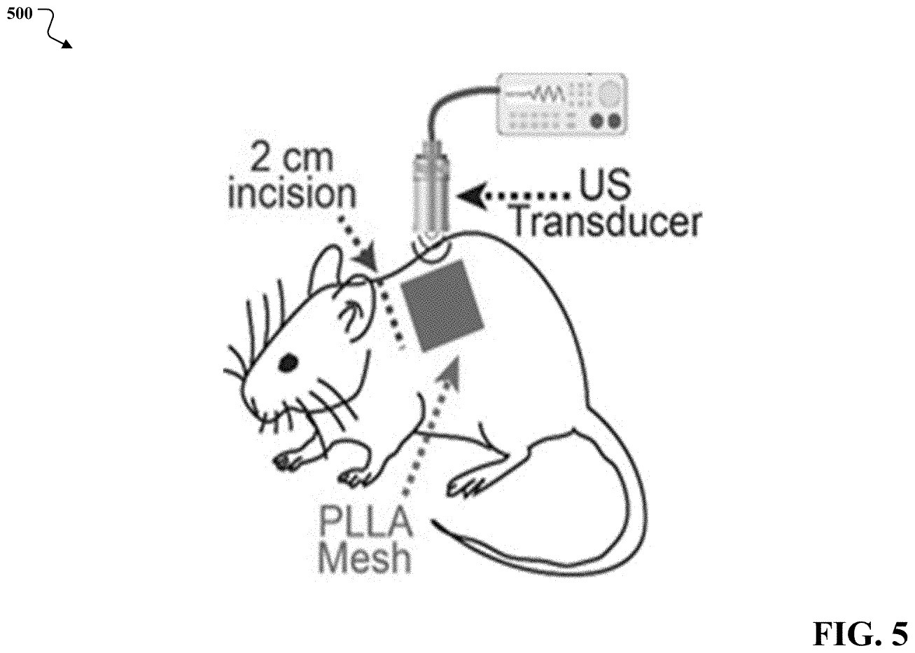

In some embodiments, the described combination therapy system breaks apart bacterial biofilms and increase cell membrane permeability for both P. aeruginosa and MRSA while inducing a broad-spectrum bactericidal effect through ROS to decrease bacterial levels so, for example, the host's immune system can fight off the antibiotic-resistant infection. In one experiment to determine the biocompatibility of the proposed combination therapy system, the effects of heat dissipation and ROS generation on mammalian tissue due to the combination therapy were measured. The 14×14 mm piezoelectric PLLA mesh was inserted into a surgically made pocket underneath the skin of a BALBc mouse. Antibiotics and analgesics were administered post-operatively. The skin over the incision was closed with 3-0 Nylon monofilament sutures. The mice were allowed to rest for 4 days prior to the commencement of their US treatment. The mice were treated for a period of 1, 2, and 3 days. For this experiment, the mouse was anesthetized with isoflurane for treatment. Then a Branson SFX150 sonifier probe was placed above the subcutaneously implanted scaffold, as illustrated in FIG. 5, which depicts a simplified schematic 500 for mice undergoing US therapy, and ultrasonic transmission gel was placed between the ultrasonic transducer and the mouse to ensure good transmission of the ultrasonic signal. The mouse was stimulated with US for 30 minutes. Twenty-four hours after their last treatment, the mouse was euthanized and the tissue surrounding the implant was extracted, immersed in formalin for 3 days, washed with water, and placed in 70% ethanol for storage. For sectioning, samples were embedded in paraffin wax at room temperature. Embedded sections were cut into 5 m thick sections and mounted on glass slides. Tissue sections were stained with hematoxylin and eosin (H&E), and Masson Trichrome stains. All sections were examined and imaged using an Olympus BX51 microscope. A sample size of (n=5) for both male and female mice were used at each time point.

In another experiment to assess the ability of the combination therapy to treat antibiotic-resistant infections in mice a 14×14 mm piezoelectric PLLA mesh was inserted into a surgically made pocket underneath the skin of a BALBc mouse. Analgesics were administered for the duration of the experiment. The skin over the incision was closed with 3-0 Nylon monofilament sutures. This experiment included five groups listed in Table 1 above and were repeated for P. aeruginosa (strain: 1077994) or S. aureus (strain: USA300-HOU-MR). For the groups, the mouse was allowed to rest for 24 hours prior to receiving a 40 μL subcutaneous injection of saline solution containing 2.40×109 CFU/mL of an antibiotic-resistant strain of bacteria (P. aeruginosa (strain: 1077994) or S. aureus (strain: USA300-HOU-MR)). The mice were allowed to rest for 3 days prior to the commencement of their treatment. The mice will be treated for a period of 1, 2, and 3 days, in accordance with their experimental group. A sample size of (n=5) for both male and female mice was used at each time point. The mice in groups 2 and 5 received US therapy and the mice were anesthetized with isoflurane for treatment. A Branson SFX150 sonifier probe was placed above the subcutaneously implanted scaffold, as illustrated in FIG. 5, and ultrasonic transmission gel was placed between the ultrasonic transducer and the mouse, to ensure good transmission of the ultrasonic signal. The mouse was stimulated with US for 30 minutes. The group 4 mice were treated with antibiotics and received a daily 0.2 ml intraperitoneal injection of saline containing a dosage of 2 milligrams (mg)/kilograms (kg) gentamicin or 7.5 mg/kg of clindamycin for treating infections with P. aeruginosa or S. aureus respectively. Twenty-four hours after the last treatment, the mice were euthanized and tissue surrounding the implant was extracted and homogenized in PBS. Then 200 μL of each homogenized tissue solution was removed and used in a dilution series where the stock solution was diluted an order of magnitude up to seven times. 5 μL of each dilution was plated in triplicate on a gridded lysogeny broth and agar plate. The plates were inverted and placed in an incubator at 37° C. for 12 hours. The colonies at the highest dilution for each group were visually counted and the remaining CFU/ml for each group were determined.

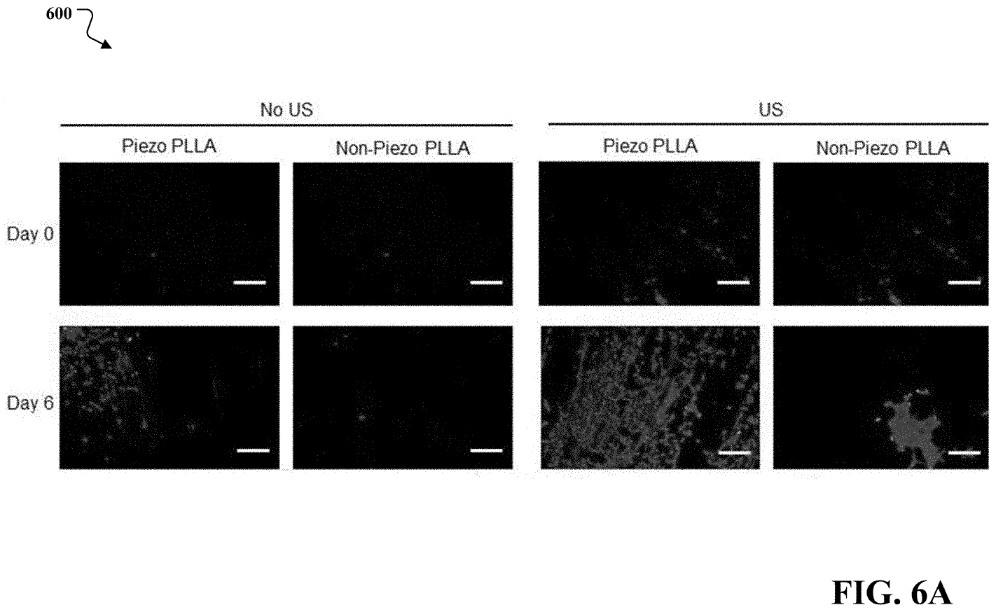

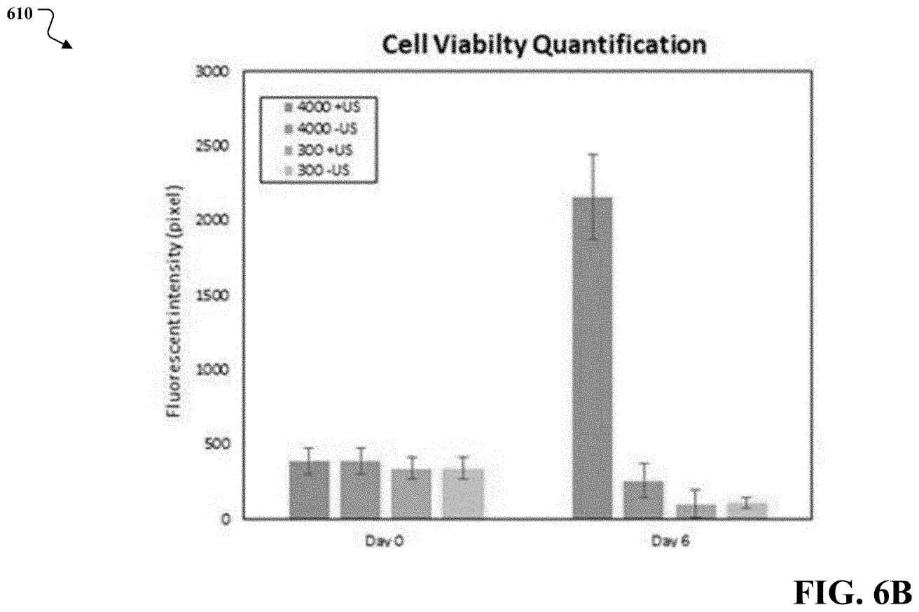

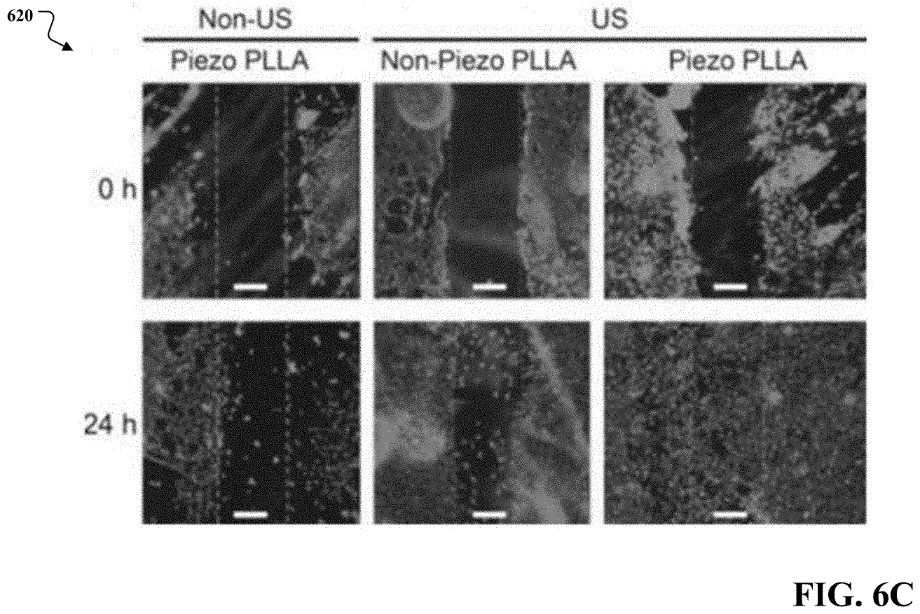

FIGS. 6A-6C depict fibroblast proliferation and migration on the piezoelectric PLLA nanofiber mesh. FIG. 6A depicts fluorescent images fibroblasts 600 on the films at day 0 and 6. The piezo film with US has the maximum cell number and density after 6 days. FIG. 6B depicts a graph 610 showing a quantitative representation of fibroblast proliferation on the films using ImageJ, significantly higher cell proliferation on the PLLA+US. FIG. 6C depicts a fibroblast migration represented by a scratch assay 620 which indicates significantly higher migration of cells into the scratch in the PLLA+US.

One experiment studied the ability of the described piezoelectric PLLA nanofiber mesh to facilitate wound healing by characterizing cell proliferation and migration on them. Mouse fibroblasts were seeded on the PLLA films and exposed to US over 1-6 days. For studying the cell proliferation, the cells were stained with a fluorescent cell membrane stain and imaged on Days 1 (soon after adhesion but before any US treatments) and Day 6 (after 6 US treatments). As seen in FIGS. 6A and 6B, the 4000 rpm sample with US showed significantly higher cell proliferation than the controls. For studying cell migration, scratch assay was performed on the PLLA films seeded with the fibroblasts, stained with a calcein-based dye, and visualized at an initial time point (after making the scratch but before any US treatments) and after 24 hours (during which they received 2 US treatments). As seen in FIG. 6C, the Piezoelectric sample with US had a much higher cell migration into the scratch area as compared to the controls. Therefore, in some embodiments, the the described combination therapy system can aid in a faster healing of the wounds in addition to treating bacterial infections at the wounded area.

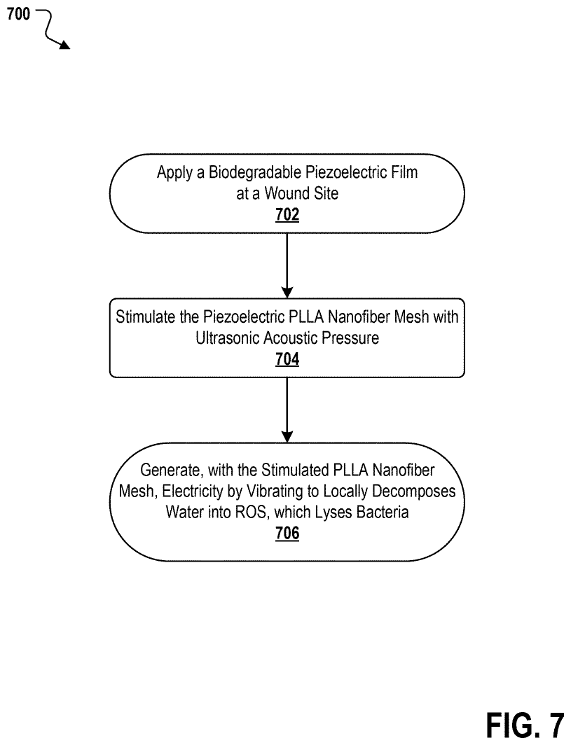

FIG. 7 depicts a flow diagram of an example process 700 for lysing bacteria at a wound site. For clarity of presentation, the description that follows generally describes the process 700 in the context of FIGS. 1-6C. However, it will be understood that the process 700 may be performed, for example, by any other suitable system or a combination of systems as appropriate.

At 702, a biodegradable piezoelectric film is applied at a wound site. The biodegradable piezoelectric film comprising a piezoelectric PLLA nanofiber mesh and the wound site compromising an electrolytically conductive environment that includes water and bacteria. In some embodiments, the electrolytically conductive environment comprise bodily fluids. In some embodiments, the PLLA nanofiber mesh comprises biodegradable piezoelectric materials that include PLLA, silk, or glycine composites. In some embodiments, wound site comprises a surgical site defect or an open wound. In some embodiments, the PLLA nanofiber mesh self-degrades after a defined lifetime. From 702, the process 700 proceeds to 704.

At 704, the piezoelectric PLLA nanofiber mesh is stimulated with ultrasonic acoustic pressure generated by an ultrasound device for prescribed period. In some embodiments, the ultrasound device stimulates the electric PLLA nanofiber mesh by generating the ultrasonic acoustic pressure continuously or pulsed. In some embodiments, the ultrasound device is configured to producing ultrasonic acoustic pressure between 36 Kilohertz (kHz) and 44 kHz. In some embodiments, the prescribed period is between 30 minutes and 120 minutes. From 704, the process 700 proceeds to 706.

At 706, the stimulated PLLA nanofiber mesh generates electricity by vibrating to locally decomposes the water into ROS, which lyses the bacteria in the electrolytically conductive environment. In some embodiments, the PLLA nanofiber mesh is configured to, when stimulated with the ultrasonic acoustic pressure, break apart to increase cell membrane permeability. In some embodiments, the generated electricity recruits cells from the electrolytically conductive environment and facilitate tissue and skin healing. From 706, the process 700 ends.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Moreover, the separation or integration of various system modules and components in the implementations described earlier should not be understood as requiring such separation or integration in all implementations, and it should be understood that the described components and systems can generally be integrated together in a single product or packaged into multiple products. Accordingly, the earlier description of example embodiments does not define or constrain this disclosure. Other changes, substitutions, and alterations are also possible without departing from the spirit and scope of this disclosure.

While the described system has been described with reference to preferred embodiments, it will be understood by those skilled in the art that various changes may be made and equivalents may be substituted for the elements thereof without departing from the scope of the disclosure. In addition, many modifications may be made to adapt the teaching of the disclosure to particular use, application, manufacturing conditions, use conditions, composition, medium, size, and/or materials without departing from the essential scope and spirit of the disclosure. Therefore, it is intended that the disclosure not be limited to the particular embodiments and best mode contemplated for carrying out this disclosure as described herein. Since many modifications, variations, and changes in detail can be made to the described examples, it is intended that all matters in the preceding description and shown in the accompanying figures be interpreted as illustrative and not in a limiting sense.

Chemical compounds are described using standard nomenclature. For example, any position not substituted by any indicated group is understood to have its valency filled by a bond as indicated, or a by hydrogen atom.

All ranges disclosed herein are inclusive of the endpoints, and the endpoints are independently combinable with each other. Each range disclosed herein constitutes a disclosure of any point or sub-range lying within the disclosed range.

The use of the terms “a” and “an” and “the” and words of a similar nature in the context of describing the improvements disclosed herein (especially in the context of the following claims) are to be construed to cover both the singular and the plural, unless otherwise indicated herein or clearly contradicted by context. Further, it should further be noted that the terms “first,” “second,” and the like herein do not denote any order, quantity, or relative importance, but rather are used to distinguish one element from another. The modifier “about” used in connection with a quantity is inclusive of the stated value and has the meaning dictated by the context (e.g., it includes, at a minimum the degree of error associated with measurement of the particular quantity).

All methods described herein can be performed in any suitable order unless otherwise indicated herein or otherwise clearly contradicted by context. The use of any and all examples, or exemplary language (e.g., “such as”), is intended merely to better illustrate the disclosure and does not pose a limitation on the scope of the disclosure or any embodiments unless otherwise claimed.

Thus, the disclosure provides, among other things, a combination therapy system that can be employed to treat infections. Various features and advantages of the disclosure are set forth in the following claims.

What is claimed is:

1. A combination therapy system for the treatment of infections, the system comprising:
an ultrasound device capable of producing ultrasonic acoustic pressure; and
a biodegradable piezoelectric film comprising polymer-based piezoelectric material, poly(L-lactic acid) (PLLA) nanofiber mesh configured to, when placed in an electrolytically conductive environment comprising water and stimulated with the ultrasonic acoustic pressure, vibrate generating electricity to locally decomposes the water into reactive oxygen species (ROS) to induce a broad-spectrum bactericidal effect.

2. The combination therapy system of claim 1, wherein the electrolytically conductive environment comprise bodily fluids.

3. The combination therapy system of claim 1, wherein the ultrasound device stimulates the electric PLLA nanofiber mesh by generating the ultrasonic acoustic pressure continuously or pulsed.

4. The combination therapy system of claim 1, wherein the piezoelectric PLLA nanofiber mesh is configured to, when stimulated with the ultrasonic acoustic pressure, break apart to increase cell membrane permeability.

5. The combination therapy system of claim 1, wherein the ultrasound device is configured to producing the ultrasonic acoustic pressure between 36 Kilohertz (kHz) and 44 kHz.

6. The combination therapy system of claim 1, wherein the ultrasound device is configured to producing the ultrasonic acoustic pressure for between 30 minutes and 120 minutes.

7. The combination therapy system of claim 1, wherein the piezoelectric PLLA nanofiber mesh comprises biodegradable piezoelectric materials that include PLLA, silk, or glycine composites.

8. The combination therapy system of claim 1, wherein the biodegradable piezoelectric film is configured to be placed at a surgical site defect or used to dress open wounds.

9. The combination therapy system of claim 1, wherein the broad-spectrum bactericidal effect includes lysing bacteria and sterilizing the area around the piezoelectric PLLA nanofiber.

10. The combination therapy system of claim 1, wherein the piezoelectric PLLA nanofiber mesh self-degrades after a defined lifetime.

11. The combination therapy system of claim 1, wherein the generated electricity recruits cells from the electrolytically conductive environment and facilitate tissue and skin healing.

12. A method for lysing bacteria at a wound site, the method comprising:
applying, at the wound site, a biodegradable piezoelectric film comprising polymer-based piezoelectric material, poly(L-lactic acid) (PLLA) nanofiber mesh, wherein the wound site comprises an electrolytically conductive environment comprising water and bacteria; and
stimulating, for a prescribed period, the piezoelectric PLLA nanofiber mesh with ultrasonic acoustic pressure generated by an ultrasound device causing the piezoelectric PLLA nanofiber mesh to generate electricity by vibrating to locally decomposes the water into reactive oxygen species (ROS) to lyse the bacteria.

13. The method of claim 12, wherein the electrolytically conductive environment comprise bodily fluids.

14. The method of claim 12, wherein the piezoelectric PLLA nanofiber mesh is configured to, when stimulated with the ultrasonic acoustic pressure, break apart to increase cell membrane permeability.

15. The method of claim 12, wherein the ultrasonic acoustic pressure is generated at between 36 Kilohertz (kHz) and 44 kHz, and wherein the prescribed period is between 30 minutes and 120 minutes.

16. The method of claim 12, wherein the piezoelectric PLLA nanofiber mesh comprises biodegradable piezoelectric materials that include PLLA, silk, or glycine composites.

17. The method of claim 12, wherein the piezoelectric PLLA nanofiber mesh self-degrades after a defined lifetime.

18. A wound dressing for therapeutic wound care comprising:
polymer-based piezoelectric material, poly(L-lactic acid) (PLLA) nanofiber mesh configured to vibrate when placed in an electrolytically conductive environment comprising water and stimulated with ultrasonic acoustic pressure, wherein the vibration of the piezoelectric PLLA nanofiber mesh within the electrolytically conductive environment generates electricity to locally decomposes the water into reactive oxygen species (ROS) to induce a broad-spectrum bactericidal effect.

19. The wound dressing of claim 18, wherein the electrolytically conductive environment comprise bodily fluids, and wherein the broad-spectrum bactericidal effect includes lysing bacteria and sterilizing the area around the piezoelectric PLLA nanofiber.

20. The wound dressing of claim 18, configured to, when stimulated with the ultrasonic acoustic pressure, break apart to increase cell membrane permeability.
